(19) United States
(12) Patent Application Publication
(10) Pub. No.: US 2016/0030184 A1
(43) Pub. Date: Feb. 4, 2016

(54) Implant with multi-directional pivoting

(71) Applicant: Leo A. Whiteside, Chesterfield, MO (US)

(72) Inventor: Leo A. Whiteside, Chesterfield, MO (US)

(73) Assignee: Signal Medical Corporation, Marysville, MI (US)

(21) Appl. No.: 14/756,749

(22) Filed: Oct. 7, 2015

Related U.S. Application Data
(62) Division of application No. 13/261,760, filed on Oct. 18, 2013.
(60) Provisional application No. 61/572,154, filed on Jul. 12, 2011.
(60) Provisional application No. 61/517,511, filed on Apr. 21, 2011.

(51) Int. Cl. A61F 2/38

(52) U.S. Cl. CPC A61F 2/385; A61F 2002/30624

(57) ABSTRACT

Implant with multi-directional pivoting has a first component including a first articular surface and a fore-to-aft and side-to-side rotation device; and a second component including a second articular surface for mating with the first articular surface and a rotation device receptacle. The first component can be mated to the second component through cooperation of the rotation device and the rotation device receptacle, and the first component can cooperate with the second component in contact of the first and second articular surfaces and in articulation of the joint when the first component is mated to the second component. The implant can be an artificial joint which generally has natural load transfer capability. In a particular embodiment, the joint is a knee prosthesis; therein, the first and second components are femoral and tibial components, with the first articular surface a femoral condylar surface and the second articular surface a tibial condylar mating surface. In such a knee, the fore-to-aft component can be considered flexion, with the side-to-side component considered version. As well, a knee prosthesis can be provided with a device to support the patella on a smooth counter surface from full extension to flexion well past 100° and/or with a mechanism to prevent excessive downward travel of the tibial component.

This is a divisional of co-pending application Ser. No. 13/261,760 filed on Oct. 18, 2013 A.D., which, as does this divisional, claims benefits as a National Stage entry of international application No. PCT/US2012/000214 filed on Apr. 20, 2012 A.D., and priority benefits of provisional application Nos. 61/517,511 filed on Apr. 21, 2011 A.D. and No. 61/572,154 filed on Jul. 12, 2011 A.D.—under the Patent Cooperation Treaty and 35 USC 119(e), 120, 363, 365 and/or 371, etc. The specifications of those applications, to include drawings, are incorporated herein by reference in their entireties.

### FIELD OF THE INVENTION

This concerns a prosthetic joint implant, for example, for a knee, which has a rotation device linking a first articular component with a second articular component, say, femoral and tibial components for the knee. It also concerns various components thereof.

### BACKGROUND TO THE INVENTION

U.S. Pat. No. 5,766,257 to Goodman et al. discloses an artificial joint having natural load transfer. The joint includes a first component having a first articular surface and a rotation device, and a second component having a second articular surface for mating with the first articular surface and a rotation device receptacle. The first component is matable to the second component through cooperation of the rotation device and the rotation device receptacle. The first component can cooperate with the second component in contact of the first and second articular surfaces and in articulation of the joint when the first component is mated to the second component. The joint can be embodied as a knee prosthesis; therein, the first and second components are femoral and tibial components, respectively, with the first articular surface being termed a condylar surface and the second articular surface being termed a condylar mating surface. The joint, especially the rotation device, may be made of metal. The joint may be implanted in suitable bone stock as a prosthesis. Compare, the RHK knee by Zimmer, Inc.

As good as that art is, a certain stiffness can remain. In particular, movement such as valgus and varus thrust otherwise encountered in a healthy joint is restricted.

It would be desirable to improve upon the art. It would be desirable to further increase mobility and natural motion in patients who have had a knee joint implant that may be considered revisional type. It would be desirable to provide the art an alternative.

### Additional Considerations or Discoveries

The femoral articular surface for the patella found with the prior art device causes excessive stress on the patellar surface from its shape and position. The tibial component, too, may travel downward excessively and may engender instability or even dislocation.

It would be desirable to address such matters, say, with a device to support the patella on a smooth counter surface from full extension to flexion well past 100° and/or with a mechanism to prevent excessive downward travel of the tibial component.

### A DISCLOSURE OF THE INVENTION

Provided, in general, is an implant with multi-directional pivoting comprising a first component including a first articular surface and a fore-to-aft and side-to-side rotation device; and a second component including a second articular surface for mating with the first articular surface and a rotation device receptacle—said first component matable to said second component through cooperation of the rotation device and the rotation device receptacle, and wherein said first component can cooperate with said second component in contact of the first and second articular surfaces and in articulation of the joint when said first component is mated to said second component. The implant can be an artificial joint which generally has natural load transfer capability. In a particular embodiment, the joint is a knee prosthesis; therein, said first and second components are femoral and tibial components, with the first articular surface being termed a femoral condylar surface and the second articular surface being termed a tibial condylar mating surface. In such a knee, the fore-to-aft component can be considered flexion, with the side-to-side component considered version. As well, a knee prosthesis can be provided with a device to support the patella on a smooth counter surface from full extension to flexion well past 100° and/or with a mechanism to prevent excessive downward travel of the tibial component. The joint implant, which, as an ensemble can be considered to be a total joint implant, may be implanted in suitable bone stock.

The invention is useful in arthroplasty.

Significantly, by the invention, the art is improved in kind. Difficulties and problems such as aforesaid are ameliorated if not overcome. The art is provided an alternative. In addition to a measure of rotation about an axis, say, a z-axis generally parallel with aligned long bones into which the total joint implant may be implanted, which would be perpendicular to an x-axis and a y-axis, the fore-to-aft and side-to-side rotation device can provide for relative motion not only in a first, fore-to-aft, plane through the x-axis but also in a second, side-to-side, plane generally perpendicular to that of the first plane, say, through the y-axis. In a particular application, in an artificial knee the problem of unnatural gait and total knee strategy has been addressed further to that addressed by Goodman et al., especially when natural bone and ligament deficiencies exist, and so forth, with mobility and natural motion in patients who have had a revisional knee joint implant enhanced to include provision or restoration at least to some degree of valgus and/or varus thrust. A more full lift, too, may be provided. Thus, the present knee joint prosthesis can have four degrees of motion or freedom, as it were, flexion, rotation, version and lift. Among other things, this additional freedom of motion of motion prevents excessive torque from being applied to the femur in the flexed position, and excessive bending stress in the extended position. Moreover, when the implant is embodied as a knee, additional patellar support can be provided, particularly through employment of an L-shaped depending member and corresponding femoral component with a reduced-length slot to accommodate the member, which allow a more full articular surface on which the patella can rest and glide in extension and flexion, thus further serving to ameliorate if not avoid unduly high compressive stress to and unnatural abrading of the patella. The additional support of the patella can reduce if not eliminate pain. As a knee, too, the implant can prevent excessive downward travel of the tibial component, which can ameliorate if not prevent dislocation or even instability. The present implant can transfer load stress in a natural manner through mating contact of its articular gliding surfaces, rather than primarily through a hinge, and closely restore the kinematics of normal anatomy to reduce risk of revisional surgery. A most highly natural articulation to the joint is provided.

Numerous further advantages attend the invention.

The drawings form part of the specification hereof With respect to the drawings, which are not necessarily drawn to scale, the following is briefly noted:

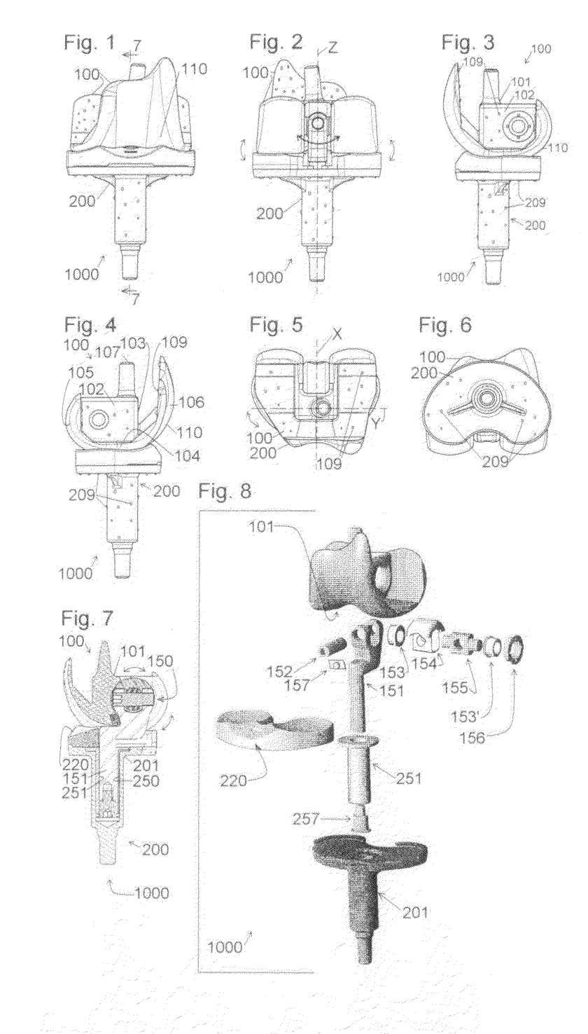

FIG. 1 is a front view (anterior to posterior) of an implant with multi-directional pivoting, embodied as a total knee joint implant ensemble. A left knee version is shown.

FIG. 2 is a rear (posterior to anterior) view of the ensemble of FIG. 1.

FIG. 3 is an outside to inside (lateral to medial) view of the ensemble of FIG. 1.

FIG. 4 is an inside to outside (medial to lateral) view of the ensemble of FIG. 1.

FIG. 5 is a top (proximal to distal) view of the ensemble of FIG. 1.

FIG. 6 is a bottom (distal to proximal) view of the ensemble of FIG. 1.

FIG. 7 is a sectional view of the ensemble of FIG. 1, taken along 7-7 of FIG. 1.

FIG. 8 is an exploded perspective view of the ensemble of FIG. 1.

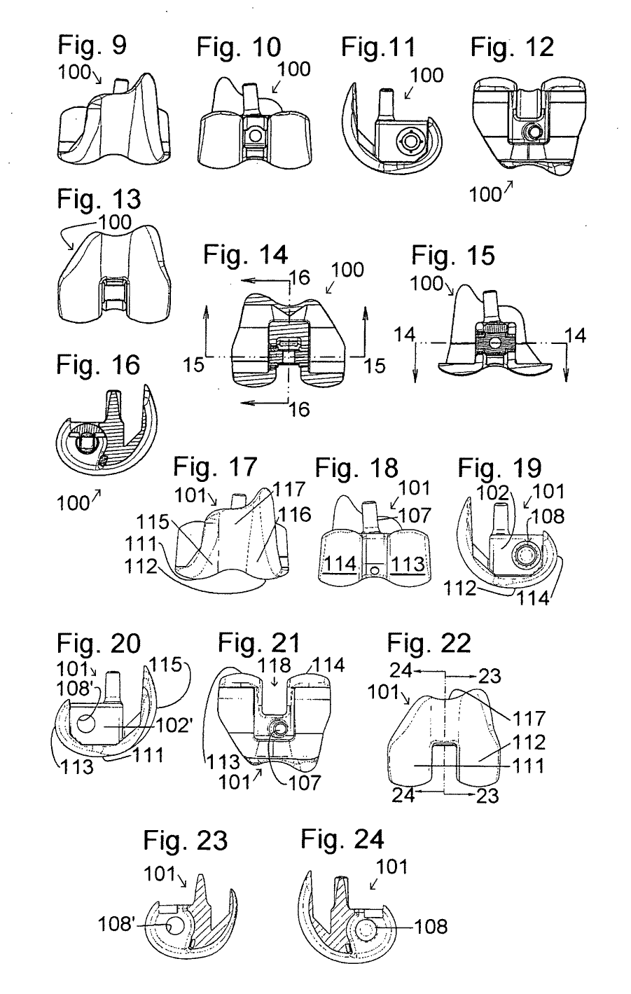

FIG. 9 is a front view (anterior to posterior) of the femoral component assembly found in the ensemble of FIG. 1, without its link, for example, as received by a surgeon.

FIG. 10 is a rear (posterior to anterior) view of the assembly of FIG. 9.

FIG. 11 is an outside to inside (lateral to medial) view of the assembly of FIG. 9.

FIG. 12 is a top (proximal to distal) view of the assembly of FIG. 9.

FIG. 13 is a bottom (distal to proximal) view of the assembly of FIG. 9.

FIG. 14 is a top (proximal to distal) sectional view of the assembly of FIG. 9, taken along 14-14 in FIG. 15.

FIG. 15 is a rear (posterior to anterior) sectional view of the assembly of FIG. 9, taken along 15-15 in FIG. 14.

FIG. 16 is an inside to outside (medial to lateral) sectional view of the assembly of FIG. 9, taken along 16-16 in FIG. 14.

FIG. 17 is a front view (anterior to posterior) of the femoral component body found in FIG. 9, without associated rotation device parts. Compare, FIGS. 7 and 8.

FIG. 18 is a rear (posterior to anterior) view of the body of FIG. 17.

FIG. 19 is an outside to inside (lateral to medial) view of the body of FIG. 17.

FIG. 20 is an inside to outside (medial to lateral) view of the body of FIG. 17.

FIG. 21 is a top (proximal to distal) view of the ensemble of FIG. 21.

FIG. 22 is a bottom (distal to proximal) view of the body of FIG. 17.

FIG. 23 is a sectional view of the body of FIG. 17, taken along 23-23 in FIG. 22.

FIG. 24 is a sectional view of the body of FIG. 17, taken along 24-24 in FIG. 22.

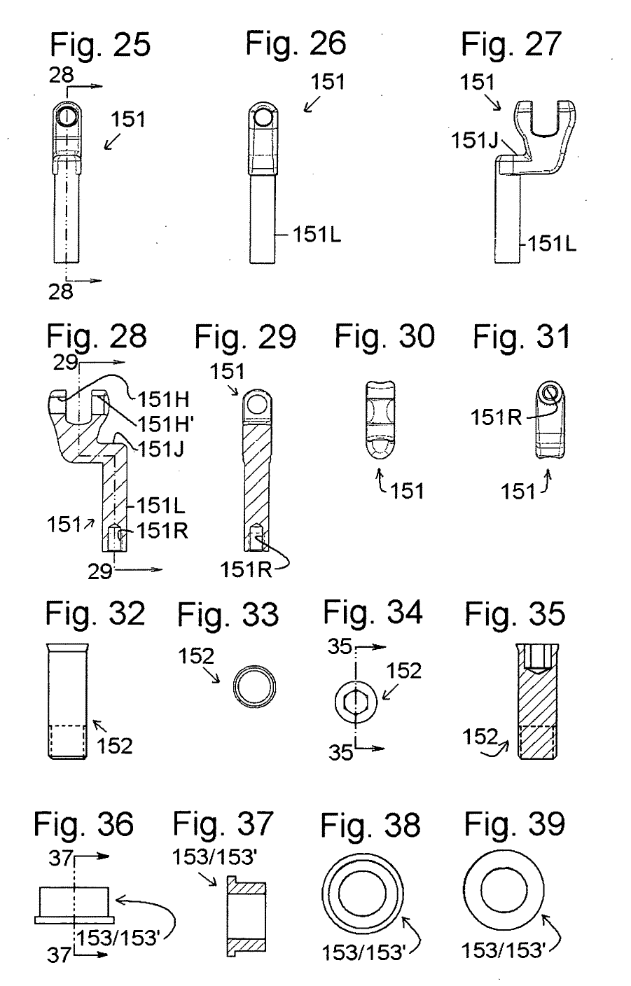

FIG. 25 is a front (anterior to posterior) view of the link found in the ensemble of FIG. 1, absent in the femoral component assembly of FIG. 9. Compare, FIGS. 7 and 8.

FIG. 26 is a rear (posterior to anterior) view of the link of FIG. 25.

FIG. 27 is an inside to outside (lateral to medial) view of the link of FIG. 25.

FIG. 28 is a sectional view of the link of FIG. 25, taken along 28-28 in FIG. 25.

FIG. 29 is a sectional view of the link of FIG. 25, taken along JOG-JOG in FIG. 28.

FIG. 30 is a top (proximal to distal) view of the link of FIG. 25.

FIG. 31 is a bottom (distal to proximal) view of the link of FIG. 25.

FIG. 32 is a side plan view of a surgeon-insertable hinge screw found in the ensemble of FIG. 1. Compare, FIGS. 7 and 8.

FIG. 33 is a rear (posterior to anterior) view of the hinge screw of FIG. 32.

FIG. 34 is a front (anterior to posterior) view of the hinge screw of FIG. 32.

FIG. 35 is a sectional view of the screw of FIG. 32, taken along 35-35 in FIG. 34.

FIG. 36 is a front or rear view of one of two hinge bearings found and employed in the ensemble of FIG. 1. Compare, FIGS. 7 and 8.

FIG. 37 is a sectional view of the bearing of FIG. 36, taken along 37-37 therein.

FIG. 38 is a superficial to deep view of the bearing of FIG. 36.

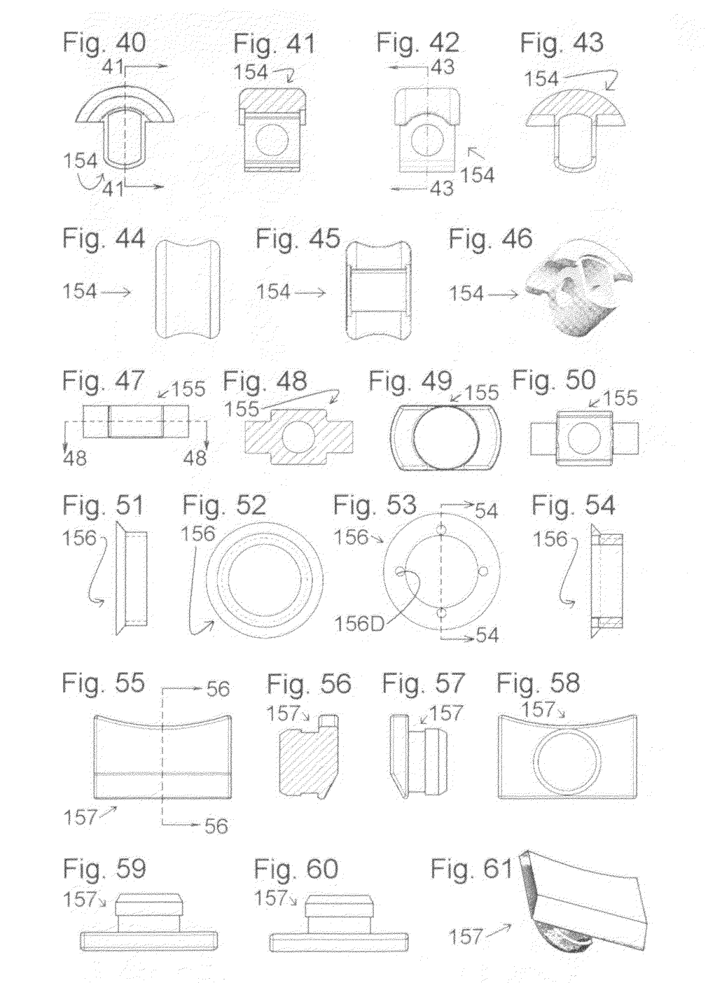

FIG. 39 is a deep to superficial view of the bearing of FIG. 36.

FIG. 40 is a side view of a hinge multi-directional pivot cover employed in the ensemble of FIG. 1. Compare, FIGS. 7 and 8.

FIG. 41 is a sectional view of the cover of FIG. 40, taken along 41-41 therein.

FIG. 42 is a front or rear view of the cover of FIG. 40.

FIG. 43 is a sectional view of the cover of FIG. 40, taken along 43-43 of FIG. 42.

FIG. 44 is a top (proximal to distal) view of the cover of FIG. 40.

FIG. 45 is a bottom (distal to proximal) view of the cover of FIG. 40.

FIG. 46 is a bottom perspective view of the cover of FIG. 40.

FIG. 47 is a top/bottom (proximal to distal or distal to proximal) view of a hinge multi-directional pivot employed in the ensemble of FIG. 1. Compare, FIGS. 7 and 8.

FIG. 48 is a sectional view of the pivot of FIG. 47, taken along 48-48 therein.

FIG. 49 is a side view of the pivot of FIG. 47.

FIG. 50 is a front/rear (anterior to posterior or posterior to anterior) view of the pivot of FIG. 47.

FIG. 51 is a front, rear, top or bottom plan view of a hinge built-in screw found in the ensemble of FIG. 1. Compare, FIGS. 7 and 8.

FIG. 52 is an inside to outside (medial to lateral) view of the screw of FIG. 51.

FIG. 53 is an outside to inside (lateral to medial) view of the screw of FIG. 51.

FIG. 54 is a sectional view of the screw of FIG. 51, taken along 54-54 in FIG. 53.

FIG. 55 is a rear (posterior to anterior) view of a bumper, which is for attachment as part of the femoral component, and which is employed in the ensemble of FIG. 1, with the orientation of view based on how the bumper is assembled in the femoral component. Compare, FIGS. 7 and 8.

FIG. 56 is a sectional view of the bumper of FIG. 55, taken along 56-56 therein.

FIG. 57 is a side view of the bumper of FIG. 55.

FIG. 58 is a front (anterior to posterior) view of the bumper of FIG. 55.

FIG. 59 is a top (proximal to distal) view of the bumper of FIG. 55.

FIG. 60 is a bottom (distal to proximal) view of the bumper of FIG. 55.

FIG. 61 is a perspective view of the bumper of FIG. 55.

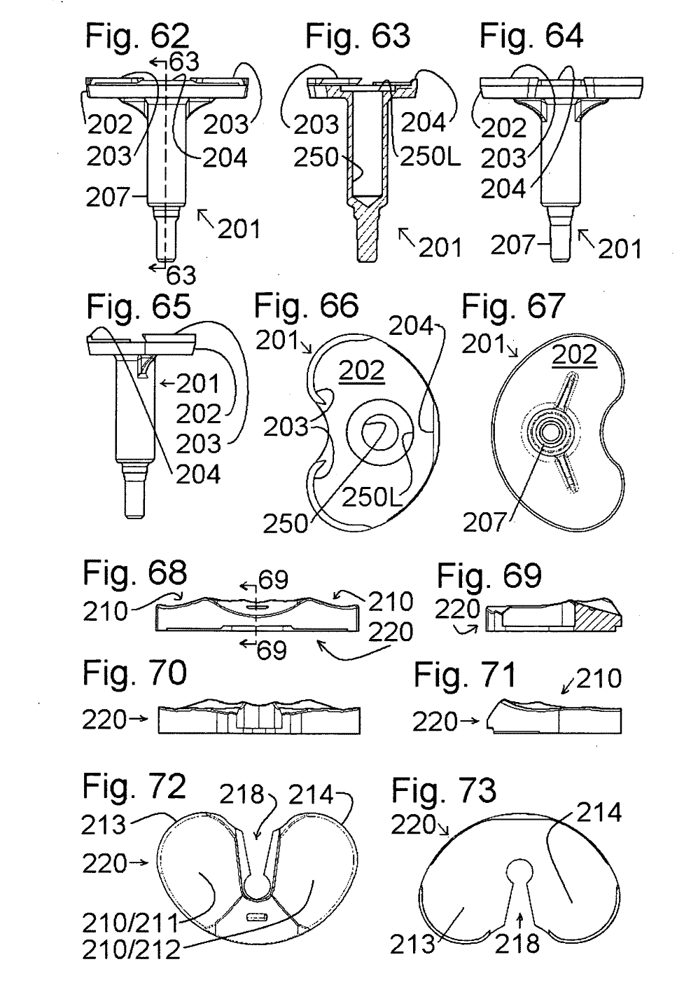

FIG. 62 is a front (anterior to posterior) view of the tibial tray body found in the ensemble of FIG. 1. Compare, FIGS. 7 and 8.

FIG. 63 is a sectional view of the tray body of FIG. 62, taken along 63-63 therein.

FIG. 64 is a rear (posterior to anterior) view of the tray body of FIG. 62.

FIG. 65 is an outside to inside (lateral to medial) view of the tray body of FIG. 62.

FIG. 66 is a top (proximal to distal) view of the tray body of FIG. 62.

FIG. 67 is a bottom (distal to proximal) view of the tray body of FIG. 62.

FIG. 68 is a front (anterior to posterior) view of the tibial tray liner found in the ensemble of FIG. 1. Compare, FIGS. 7 and 8.

FIG. 69 is a sectional view of the liner of FIG. 68, taken along 69-69 therein.

FIG. 70 is a rear (posterior to anterior) view of the liner of FIG. 68.

FIG. 71 is an outside to inside (lateral to medial) view of the liner of FIG. 68.

FIG. 72 is a top (proximal to distal) view of the liner of FIG. 68.

FIG. 73 is a bottom (distal to proximal) view of the liner of FIG. 68.

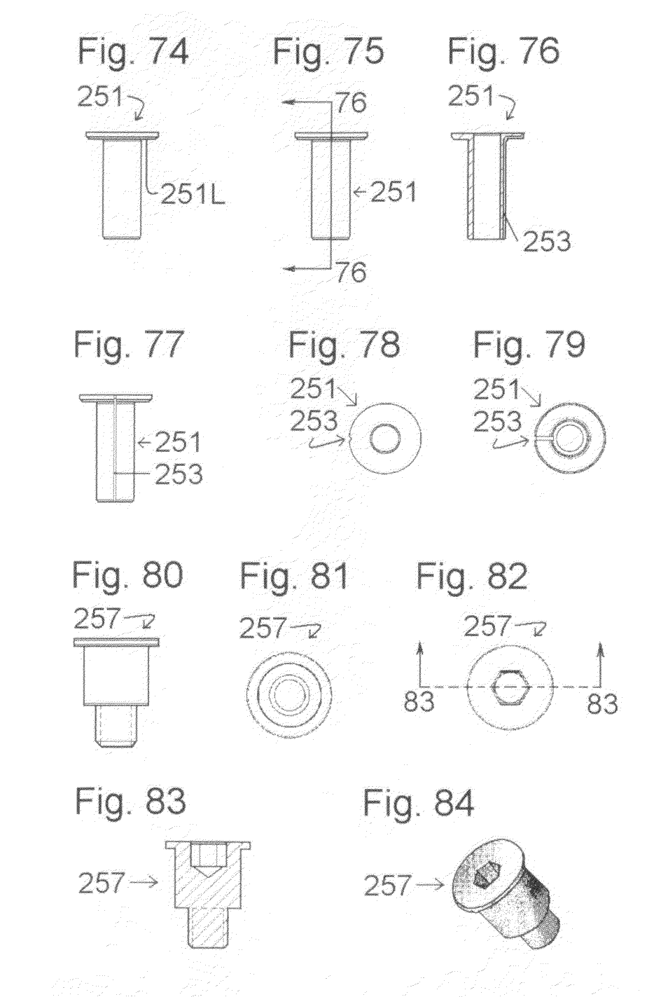

FIG. 74 is a side view of a link receptacle liner employed in the ensemble of FIG. 1. Compare, FIGS. 7 and 8.

FIG. 75 is another side view of the receptacle liner of FIG. 74, at a 90-degree angle to the side view in FIG. 74.

FIG. 76 is a sectional view of the receptacle liner of FIG. 74, taken along 76-76 in FIG. 75.

FIG. 77 is another side view of the receptacle liner of FIG. 74, at a 180-degree angle to the side view of FIG. 75.

FIG. 78 is a top (proximal to distal) view of the receptacle liner of FIG. 74.

FIG. 79 is a bottom (distal to proximal) view of the receptacle liner of FIG. 74.

FIG. 80 is a side plan view of an anti-lift screw employed in the ensemble of FIG. 1. Compare, FIGS. 7 and 8.

FIG. 81 is a top (proximal to distal) view of the screw of FIG. 80.

FIG. 82 is a bottom (distal to proximal) view of the screw of FIG. 80.

FIG. 83 is a sectional view of the screw of FIG. 80, taken along 83-83 in FIG. 82.

FIG. 84 is a bottom perspective view of the screw of FIG. 80.

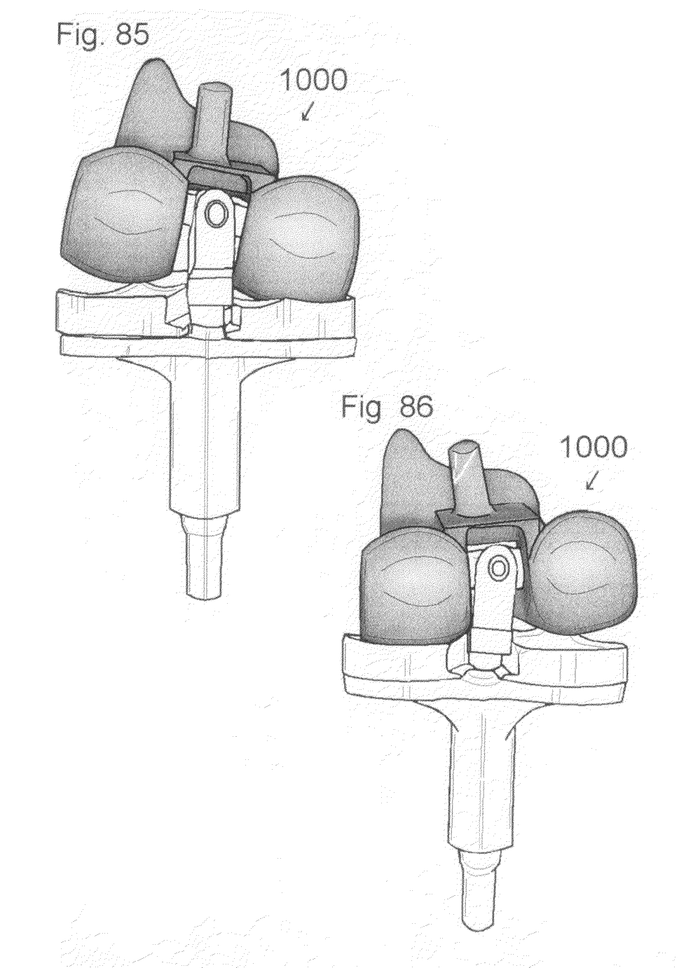

FIG. 85 is a rear (posterior to anterior) view of the ensemble of FIG. 1, in a position of valgus thrust.

FIG. 86 is a rear (posterior to anterior) view of the ensemble of FIG. 1, in a position of varus thrust.

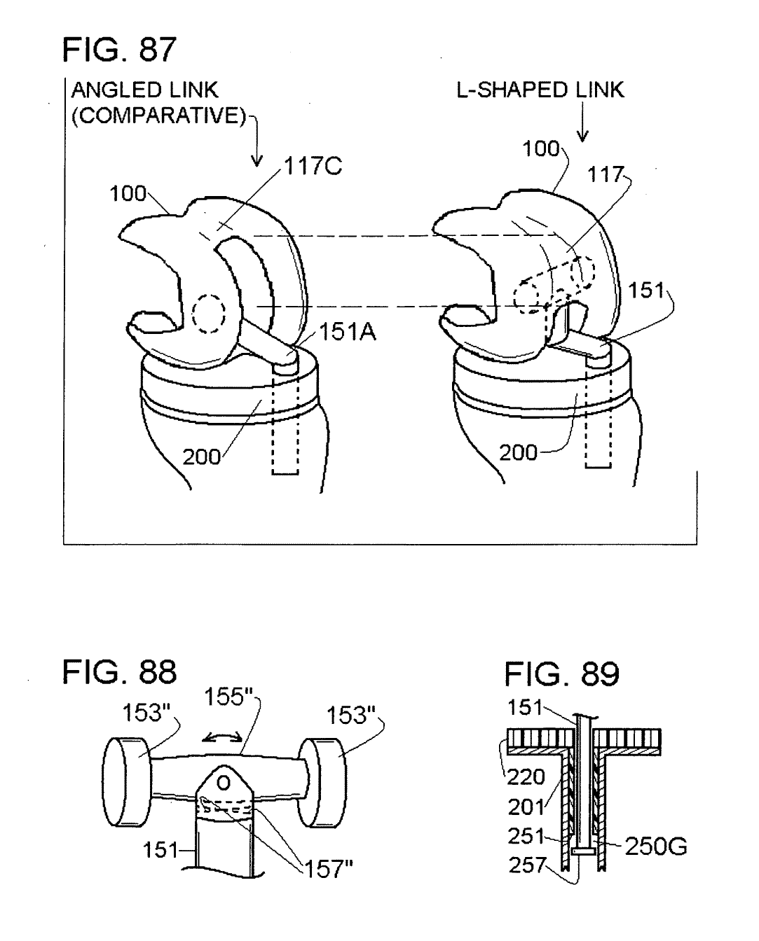

FIG. 87 is a side-by-side view of a knee implant as a comparative having an angled depending member connecting femoral and tibial parts versus a knee implant improvement having a device to support the patella through a more broad range of flexion and extension with employment of an L-shaped depending link.

FIG. 88 is a front (anterior to posterior) view of a portion of a fore-to-aft and side-to-side rotation device as may be found in a knee implant improvement as in FIG. 87.

FIG. 89 is a front (anterior to posterior) sectional view of a portion of a tibial component with a depending link inserted therein.

The invention can be further understood by the detail set forth below, which may be read in view of the drawings. The following, as with the foregoing, should be taken in an illustrative and not necessarily limiting sense.

The instant implant includes multi-directional pivoting capability. As a total joint implant it can embrace an ensemble having two main components, the first including a first articular surface and a fore-to-aft and side-to-side rotation device, and the second including a second articular surface for mating with the first articular surface and a rotation device receptacle. The first component can be mated to the second component through cooperation of the rotation device and the rotation device receptacle, and can cooperate with the second component with contact of its articular surface with the second articular surface and articulation of the joint when mated to the second component. The rotation device provides for not only fore-to-aft pivoting such as in an x-z plane but also side-to-side pivoting such as in a y-z plane. Further, the rotation device in conjunction with the corresponding receptacle, for example, through incorporation of radially symmetric geometry such as that of a cylinder, cone or truncated cone with respect to corresponding registering or mating surfaces of a link of the rotation device with the receptacle, or through an undersized link in a receptacle where either of both of the same are not so radially symmetric, may provide for pivoting such as about the z-axis. A yoke and axle mechanism can achieve the bi-axial rotation of the femoral joint; this allows the femoral shaft to be fixed to the femoral component prior to insertion in the bore, and the tibial component to be linked to the femoral component through an assembly step. Moreover, the instant implant can have an offset connecting member, for example, as an L-shaped member, between the femoral rotational joint and the tibial rotational joint to allow the articular surface to be elongated posteriorly for enhanced patellar support.

The implant with multi-directional pivoting can be made with any suitable material(s), which may be biocompatible. For instance, metal(s) and/or alloy(s) can be employed, with or without employment of other material(s) such as plastic(s), say, for certain bearing(s) and/or articulation insert(s). Ceramic(s) and/or composite(s) may be employed itself or themselves or in conjunction with other material(s). The metal(s) and/or alloy(s), ceramic(s) and/or composite(s) may be coated with another material, say, a metal or alloy coating a ceramic or composite, or vice versa. A magnesium oxide tetragonally toughened zirconia (Mg-TTZ) ceramic may be employed such as disclosed by Serafin, Jr. et al., Pub. No. US 2006/0025866 A1. The Mg-TTZ or part thereof may be coated with a metal or alloy, say, commercially pure titanium (CPT) as a CPT porous coat, such as disclosed by Serafin, Jr. et al., Pub. No. US 2010/0076566 A1.

The instant implant can be made by any suitable method or process. Thus, casting, molding, forging, computer numeric control (CNC) machining and/or polishing can be employed. See also, the aforementioned '866 and '566 publications.

With more particular reference to the drawings, implant with multi-directional pivoting 1000 is embodied as a total knee joint prosthesis, depicted for the left human knee. The implant 1000 includes first component 100, which is a femoral component, and which includes first articular surface 110 that can be termed a femoral condylar surface, and fore-to-aft and side-to-side rotation device 150; and second component 200, which is a tibial component, and which includes second articular surface 210 that can be termed a tibial condylar mating surface, and rotation device receptacle 250.

The femoral component 100 includes femoral component body 101, typically of a one-piece construction of a suitable substance such as of metal, ceramic, engineering plastic or composite. For instance, a cobalt alloy, for example, CoCr, which may conform to ASTM F75, F799 or F1537, may be employed. The frame 101 can include side walls 102, 102′; front wall 103; distal condylar flange 104; posterior flange 105; anterior flange 106; femoral bone stock insertion stem 107; and wall holes 108, 108′ for rotation device 150. Porous, interiorly-facing surface 109 may face in proximal and deep directions. The first articular surface of the femoral component, i.e., condylar surface 110, of generally convex geometry, generally includes inferior, medial condyle; inferior, lateral condyle 112; posterior, medial condyle 113; posterior, lateral condyle 114, and may be considered to include anterior, medial condyle 115, and anterior, lateral condyle 116, although much if not all of the anterior condyles 115 and 116 are restrained from coming into contact with the condylar mating surface 210. On the superficial side of the anterior flange 106 can be provided trochlear surface 117, i.e., trochlea, on which the actual or an artificial patella may generally ride. Inter-condylar notch 118 can be formed. Typically the condylar surface 110 (which includes the condyles 111-116) and the trochlea 117 are smooth and highly polished. The fore-to-aft and side-to-side rotation device 150 includes offset depending link 151, which, for example, may be of cobalt-chrome alloy, and which may have hinge holes 151H, 151H′, which hole 151H may be threaded, and distal to jog 151J radially symmetric, smooth lower leg 151L and receptacle 151R that may be threaded; surgeon-insertable hinge screw 152, say, of cobalt-chrome alloy, which passes through the holes 151H, 151H′ and is threaded on the threads about the hole 151H, and which provides for attachment of the link 151 and pivoting through the y-axis so as to provide for valgus and varus thrust motion, with the radially symmetric lower leg 151L made to provide rotation about the z-axis in use; hinge bearings 153, 153′, which, for example, may be of ultra high molecular weight polyethylene (UHMWPE) such as would conform to ASTM F648, be identical in size and configuration, and fit snugly in the respective holes 108, 108′ of the side walls 102, 102′; hinge multi-directional pivot cover 154, for example, of UHMWPE; hinge multi-directional pivot 155, which, for example, may be of cobalt-chrome alloy, and which can pass through the holes in the hinge bearings 153, 153′ and provide for pivoting through the x-axis so as to provide flexion and extension; hinge built-in screw 156, which, for example, of cobalt-chrome alloy, and which may assist in holding the pivot 155 and associated assembly in place by being threaded into the wall 102, say using threading assist dents 156D for the purpose; and bumper 157, which, for example, may be of UHMWPE, and which is attached by a mechanical locking mechanism and so forth and the like, and functions so as to dampen the extension limit of the assembly. Order of insertion may vary from that set forth above.

The tibial component 200 can include tibial component tray body 201, typically of a one-piece construction of a suitable substance such as of metal, ceramic, engineering plastic or composite. For instance, a titanium alloy, for example, Ti 6Al—4V, which may conform to ASTM F67, F136 or F1472, may be employed. The body 201 can include tibial tray 202 having a set of liner-engaging, lipped walls 203; liner-stopping block 204; and stem 207. Porous, interiorly-facing surface 209, for example, on the underside of the tray 202 and proximal portion of the stem 207 may face in distal and superficial directions, respectively. The second articular surface of the tibial component, i.e., condylar mating surface 210, which is generally of concave geometry in relation to the convex geometry of the condylar surface 110, generally includes superior, medial articular surface 211 and superior, lateral articular surface 212 on medial lobe 213 and lateral lobe 214, respectively, which provide for inter-condylar notch 218 analogous to the inter-condylar notch 118. The notch 218 can allow for insertion around the offset depending link 151. Such features 210-214 and 218 may be provided on a separable tibial tray liner 220 of suitable material, for example, UHMWPE. Through the top of the tibial tray 202 and into the tibial component stem 207 is provided the rotation device receptacle 250, which may be, for example, in the form of an essentially cylindrical cup having top shoulder countersink lip 250L. Rotation device receptacle liner 251, for example, of UHMWPE, can be inserted into the receptacle 250 so as to itself receive the lower leg 151L of the link 151. The liner 251 can be internally radially symmetrical to correspond with the lower leg 151L and can include shoulder lip 251L, which can fit in the rotation device receptacle top shoulder countersink lip 250C; and axially directed groove 253 to permit exit of entrained body fluids, say, between the inter-condylar notches 118, 218 during front to back extension and flexion and side to side valgus and varus thrust of the implanted joint 1000 and consequent up and down motion of the rotation device link 151, which fits quite closely although movably within the liner 251, and during insertion of the link 151 into the liner 251 during implantation. Restraining screw 257, say, which may be of biocompatible metal, attaches to the receptacle 151R and can engage a lower extremity of the liner 251 to keep the link 151, hence the femoral component 100, from pulling away from the tibial component 200 in use, thus providing a mechanism to prevent excessive downward travel of the tibial component 200 as when the implant 1000 is implanted, so, more generically, to prevent excess travel between a first component and a second component such that the second component cannot travel apart and separate to an unnaturally large degree from the first component when the implant is implanted, as in a body of a patient.

Accordingly, in general, not only is rotation about a z-axis and fore-to-aft and side-to-side motions in x and y axes provided, which in an artificial knee such as that of the patent to Goodman et al. can include provision or restoration at least to some degree of valgus and/or varus thrust, and a more full lift, which things of themselves are highly significant achievements, but even further advantages are provided. Thus, by way of explanation through examples and further disclosures of certain of these, in the normal knee the longitudinal axis of the tibia is displaced anteriorly relative a transverse axis through which the tibia rotates during flexion and extension. In accommodating this, a connecting member connects an axle through which the tibia rotates during flexion and extension to one through which it rotates longitudinally during internal and internal rotation. This connecting member may be angled such as illustrated with angled depending link 151A, but that requires a large notch in the front of the femoral component, i.e., a large slot in patellar support portion of the femoral component, to allow it to move from flexion to full extension, which takes up much of the patellar support articular surface leaving a comparatively small trochlear articular surface 117C to support a patella, and thus applies high compressive stress to the patella and also abrades it. This issue is addressed with an L-shaped connecting member such as the offset depending link 151 and so forth, which avoids what would be much of an upper portion of such a large notch and comparatively small trochlear articular surface 117C as mentioned above and permits instead employment there of material that can function as part of a now extensive trochlear articular surface 117 of the femoral component 100 to support the patella. Compare, FIG. 87. This results in a “patella-friendly” femoral component that supports the patella in deep flexion, and, thus, the L-shaped depending link 151 allows full extension and flexion with patellar support. Thus, a device to support the patella on a smooth counter surface from full extension to flexion past 100°, represented by the trochlear articular surface 117, is provided. Moreover, the mechanism that provides connection of an L-shaped connecting member, which may include a link such as the link 151, to a transverse axle, which may include the hinge screw 152 in the mechanism of FIGS. 1-86 or an alternative such as axle 155″ having bearings 153″ depicted within the mechanism in FIG. 88, allows it to be assembled in the knee with the bearing assembled to the femoral component 100. This allows the femoral component 100 to be pre-assembled with its bearings 153″ and axle 155″ prior to insertion in the femur, which saves bone by alleviating the process of inserting the axle from the side, and an additional advantage of such a mechanism is that side-to-side (varus-valgus) laxity of the knee is constrained both by the limited tilt of the mechanism of the axle and the limited pistoning mechanism inside the tibial component. Thus, tilting side-to-side can be constrained by impingement, say, along boundary 157″. See also, FIG. 89, where, among other things, such a tibial component allows limited slip and helps limited tilt of the corresponding femoral component. Gap 250G may accommodate upward and downward movement with the link 151 and so forth.

The present invention is thus provided. Various feature(s), part(s), subcombination(s) and/or combination(s) can be employed with or without reference to other feature(s), part(s), subcombination(s) and/or combination(s) in the practice of the invention, and numerous modifications can be effected within its spirit, the literal claim scope of which is particularly pointed out as follows:

What is claimed is:

1. In a total knee joint implant prosthesis having femoral and tibial components and a connecting member therebetween, with the femoral component including a femoral condylar surface and the tibial component including a tibial condylar mating surface, the improvement which comprises the prosthesis being provided with a device to support the patella on a smooth counter surface from full extension to flexion past 100° and/or with a mechanism to prevent excessive downward travel of the tibial component when implanted in a patient.

2. The prosthesis of claim 1, wherein patellar support is provided through employment of an L-shaped depending link as the connecting member and a corresponding femoral component with a slot to accommodate the depending link, which allow a substantially extended trochlear articular surface on which the patella can rest and glide in extension and flexion, which results in a femoral component that can support the patella in deep flexion, thus further serving to ameliorate if not avoid unduly high compressive stress to and unnatural abrading of the patella.

3. The prosthesis of claim 2, wherein the L-shaped depending link is of one and only one piece, and pivots about a first, proximal end of the link; includes a radially symmetric lower leg on an opposing end of the link, which is received by a rotation device receptacle of the tibial component; and has a jog therebetween.

4. The prosthesis of claim 1, wherein both said device to support the patella and said mechanism are present.

5. The prosthesis of claim 2, wherein both said device to support the patella and said mechanism are present.

6. The prosthesis of claim 3, wherein both said device to support the patella and said mechanism are present.

7. The prosthesis of claim 1, wherein said mechanism includes a restraining screw, which attaches to a distal end of the offset L-shaped depending link.

8. The prosthesis of claim 2, wherein said mechanism includes a restraining screw, which attaches to a distal end of the offset L-shaped depending link.

9. The prosthesis of claim 3, wherein said mechanism includes a restraining screw, which attaches to a distal end of the offset L-shaped depending link.

10. The prosthesis of claim 4, wherein said mechanism includes a restraining screw, which attaches to a distal end of the offset L-shaped depending link.

11. The prosthesis of claim 5, wherein said mechanism includes a restraining screw, which attaches to a distal end of the offset L-shaped depending link.

12. The prosthesis of claim 6, wherein said mechanism includes a restraining screw, which attaches to a distal end of the offset L-shaped depending link.
